(19) United States
(12) Patent Application Publication
(10) Pub. No.: US 2021/0330606 A1
(43) Pub. Date: Oct. 28, 2021

(54) GLUCOSE CONSUMPTION ACCELERATOR AND GLYCOLYSIS ACCELERATOR

(71) Applicants: Kenji SORIMACHI, Takasaki-shi, Gunma (JP); SHINKO SANGYO Co., Ltd., Takasaki-shi, Gunma (JP)

(72) Inventor: Kenji SORIMACHI, Gunma (JP)

(21) Appl. No.: 16/610,360

(22) PCT Filed: May 27, 2019
(86) PCT No.: PCT/JP2019/020826

§ 371 Date: Nov. 1, 2019

(30) Foreign Application Priority Data
Jul. 31, 2018 (JP) 2018-143056
Sep. 27, 2018 (JP) 2018-181302
Feb. 4, 2019 (JP) PCT/JP2019/003915

(51) Int. Cl. A61K 31/133; A61K 31/535; A61K 31/137; A61K 31/136; A61K 31/131; A61K 9/00; A61P 3/10

(52) U.S. Cl. CPC A61K 31/133; A61K 31/535; A61K 31/137; A61P 3/10; A61K 31/131; A61K 9/0053; A61K 31/136

(57) ABSTRACT

The present invention provides a new drug that can accelerate glucose consumption. The glucose consumption accelerator of the present invention includes: at least one selected from the group consisting of 2-amino-1-cyclohexylethanol, 1-amino-2-propanol, 1,3-bis[tris(hydroxymethyl)methylamino]propane, N-cyclohexylethanolamine, diethanolamine, diethylamine, dipropylamine, morpholine, propylamine, triethanolamine, triethylamine, and trishydroxymethylaminomethane.

### TECHNICAL FIELD

The present invention relates to a glucose consumption accelerator and a glycolysis accelerator.

### BACKGROUND ART

Diabetes is a disease in which insulin does not work adequately, so that the glucose concentration (blood glucose level) in the blood continues to be high. It is known that long-term diabetes causes vascular damage due to high concentration of glucose, resulting in complications such as heart disease, blindness, renal failure, and amputation of the foot.

As a method for the treatment of diabetes, for example, there is known a method of controlling blood glucose level by replenishment of insulin from outside the body by insulin injection in the case of type 1 Diabetes, and by drug therapies such as oral hypoglycemic drugs and insulin injection, in addition to diet therapy and exercise therapy, in the case of type 2 Diabetes (Patent Literature 1). However, there is a need for new methods that can control glucose concentration.

### CITATION LIST

### Patent Literature

### Patent Literature

Patent literature 1: JP 2015-205925 A

### SUMMARY OF INVENTION

### Technical Problem

Accordingly, it is an object of the present invention to provide a new drug that can accelerate the consumption of glucose.

### Solution to Problem

In order to achieve the above object, the present invention provides a glucose consumption accelerator and a glycolysis accelerator each including: at least one selected from the group consisting of 2-amino-1-cyclohexylethanol, 1-amino-2-propanol, 1,3-bis[tris(hydroxymethyl)methylamino]propane, N-cyclohexylethanolamine, diethanolamine, diethylamine, dipropylamine, morpholine, propylamine, triethanolamine, triethylamine, and trishydroxymethylaminomethane.

The present invention also provides a pharmaceutical composition for use in treatment of diabetes, including: the glucose consumption accelerator or the glycolysis accelerator according to the present invention.

The present invention also provides a glucose consumption accelerating method, including the step of: administering the glucose consumption accelerator or the pharmaceutical composition according to the present invention. The present invention also provides a glycolysis accelerating method, including the step of: administering the glycolysis accelerator or the pharmaceutical composition according to the present invention.

### Advantageous Effects of Invention

According to the present invention, a new drug that can accelerate the glucose consumption can be provided.

### BRIEF DESCRIPTION OF DRAWINGS

FIGS. 1A and 1B are graphs each showing relative values of glucose consumption of fibroblasts to which the drugs were added in Example 1.

FIGS. 2A and 2B are graphs each showing relative values of glucose consumption of fibroblasts to which the drugs of the comparative example were added in Example 1.

FIGS. 3A and 3B are graphs each showing relative values of glucose consumption of fibroblasts to which the drugs were added in Example 2.

FIG. 4 is a graph showing relative values of glucose consumption of fibroblasts to which the drugs were added in Example 3.

FIG. 5 is a graph showing relative values of glucose consumption of fibroblasts to which the drugs were added in Example 4.

FIG. 6 is a graph showing relative values of glucose consumption of liver cancer cells to which the drugs were added in Example 5.

FIGS. 7A and 7B are graphs each showing relative values of lactate concentration of fibroblasts and liver cancer cells to which the drugs were added in Example 6.

FIGS. 8A and 8B are graphs showing blood glucose levels of mice to which the drug was administered in Example 7.

FIGS. 9A and 9B are graphs each showing calculated values of AUC of the mice to which the drug was administered in Example 7.

FIG. 10 is a graph showing the lactate concentrations in the culture solution of each cell to which the drug was administered in Example 8.

FIG. 11 is a graph showing the lactate concentration in the culture solution of each cell to which the drug was administered in Example 9.

### DESCRIPTION OF EMBODIMENTS

The glucose consumption accelerator and the glycolysis accelerator of the present invention include, for example, at least one selected from the group consisting of 2-amino-1-cyclohexylethanol, 1-amino-2-propanol, 1,3-bis[tris(hydroxymethyl)methylamino]propane, N-cyclohexylethanolamine, diethanolamine, diethylamine, dipropylamine, morpholine, propylamine, triethanolamine, triethylamine, and trishydroxymethylaminomethane as a main component.

The glucose consumption accelerator and the glycolysis accelerator of the present invention further include, for example, an oral additive.

The terms used in the present specification can be used in the meaning commonly used in the art unless otherwise specified.

The present invention is described below in details.

(Glucose Consumption Accelerator and Glycolysis Accelerator)

As described above, the glucose consumption accelerator and the glycolysis accelerator of the present invention are characterized in that they contain at least one selected from the group consisting of the compound represented by the following chemical formula (1), tautomers and stereoisomers thereof, and salts thereof (hereinafter also referred to as “drugs of the present invention”). In the glucose consumption accelerator and the glycolysis accelerator of the present invention, other constitution and conditions are not particularly limited.

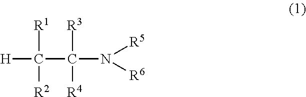

In the chemical formula (1), R¹ is a hydrogen atom or a hydroxyl group. Also, as described below, R¹ and R⁶ may together form a ring structure.

In the chemical formula (1), R² is a substituent containing a hydrogen atom, a straight-chain or branched alkyl group, an aryl group, a cycloalkyl group, or a heterocycle. The carbon number of the straight-chain or branched alkyl group is not particularly limited, and may be, for example, 1 to 40, 1 to 32, 1 to 24, 1 to 18, 1 to 12, 1 to 6, or 1 to 2 (2 or more in the case of an unsaturated hydrocarbon group). Specific examples of the alkyl group include a methyl group, an ethyl group, an n-propyl group, an isopropyl group, an n-butyl group, an isobutyl group, a sec-butyl group, a tert-butyl group, a pentyl group, a hexyl group, a heptyl group, an octyl group, a nonyl group, a decyl group, an undecyl group, a dodecyl group, a tridecyl group, a tetradecyl group, a pentadecyl group, a hexadecyl group, a heptadecyl group, an octadecyl group, a nonadecyl group, and an icosyl group. The same applies to groups (hydroxyalkyl groups) derived from alkyl groups. The aryl group includes, for example, a monocyclic aromatic hydrocarbon group and a polycyclic aromatic hydrocarbon group. The monocyclic aromatic hydrocarbon group can be, for example, phenyl. Examples of the polycyclic aromatic hydrocarbon group include a 1-naphthyl group, a 2-naphthyl group, a 1-antril group, a 2-antril group, a 9-antril group, a 1-fenantril group, a 2-fenantril group, a 3-fenantril group, a 4-fenantril group, and a 9-fenantril group. Examples of the cycloalkyl group include a cyclopropyl group, a cyclobutyl group, a cyclopentyl group, a cyclohexil group, a cycloheptyl group, and a cyclooctyl group. The number of atoms constituting the ring in the heterocycle in the substituent containing the heterocycle is not particularly limited, and is, for example, 3, 4, 5, 6, 7, 8, 9, or 10. The heteroatom in the substituent containing the heterocycle is, for example, at least one selected from the group consisting of O, S, N, and NH. Specifically, the substituent containing the heterocycle can be, for example, a group represented by the following chemical formula (R2). In the following chemical formula (R2), the atoms constituting the ring may be CH instead of N, O instead of S, or S instead of O.

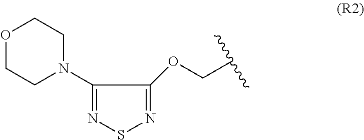 [SMILES: *COc1nsnc1N1CCOCC1]

When a substituent or the like has isomers, any isomer can be used, unless otherwise stated.

One or more of the hydrogen atoms bonded to the carbon atoms of the substituent including the alkyl group, the aryl group, the cycloalkyl group, and the heterocycle may be substituted with another substituent. Such a substituent is not particularly limited, and examples thereof include carboxy, halogen, alkyl halide (e.g., CF₃, CH₂CF₃, CH₂CCl₃), nitro, nitroso, cyano, alkyl (e.g., methyl, ethyl, isopropyl, tert-butyl), alkenyl (e.g., vinyl), alkynyl (e.g., ethinyl), cycloalkyl (e.g., cyclopropyl, adamantyl), cycloalkyl alkyl (e.g., cyclohexylmethyl, adamantylmethyl), cycloalkenyl (e.g., cyclopropenyl), aryl (e.g., phenyl, naphthyl), arylalkyl (e.g., benzyl, phenethyl), heteroaryl (e.g., pyridyl, furyl), heteroarylalkyl (e.g., pyridylmethyl), heterocyclyl (e.g., piperidyl), heterocyclylalkyl (e.g., morpholyl methyl), alkoxy (e.g., methoxy, ethoxy, propoxy, butoxy), perfluoroalkyl (e.g., CF₃), halogenated alkoxy (e.g., OCF₃), acyl, alkenyloxy (e.g., vinyloxy, aryloxy), aryloxy (e.g., phenyloxy), alkyloxycarbonyl (e.g., methoxycarbonyl, ethoxycarbonyl, tert-butoxycarbonyl), arylalkyloxy (e.g., benzyloxy), amino[alkylamino (e.g., methylamino, ethylamino, dimethylamino), acylamino (e.g., acetylamino, benzoylamino), arylalkylamino (e.g., benzylamino, tritylamino), hydroxyamino], alkylaminoalkyl (e.g., diethylaminomethyl), sulfamoyl, and oxo.

In the chemical formula (1), R³, R⁴, and R⁵ are each a hydrogen atom, a straight-chain or branched alkyl group, or a straight-chain or branched hydroxyalkyl group. The straight-chain or branched alkyl group and the straight-chain or branched hydroxyalkyl group are, for example, as described above. R³, R⁴, and R⁵ may be identical to or different from each other. Also, as described below, R⁵ and R⁶ may together form a ring structure. One or more of the hydrogen atoms bonded to the carbon atoms of the alkyl group and the hydroxyalkyl group may be substituted with another substituent. Examples of such a substituent are as described above.

In the chemical formula (1), R⁶ is a substituent containing a hydrogen atom, a straight-chain or branched alkyl group, a straight-chain or branched hydroxyalkyl group, a cycloalkyl group, or a heteroatom, and may or may not be straight-chain or branched, and may or may not contain a ring structure. The straight-chain or branched alkyl group, the straight-chain or branched hydroxyalkyl group, and the cycloalkyl group are, for example, as described above. The number of carbon atoms of the substituent containing the heteroatom is not particularly limited, and may be, for example, 1 to 40, 1 to 32, 1 to 24, 1 to 18, 1 to 12, 1 to 6, or 1 to 2. The heteroatom in the substituent containing the heteroatom is, for example, at least one selected from the group consisting of O, S, N, and NH. Specific examples of the substituent containing the heterocycle include groups represented by the following chemical formulae (R6-1) and (R6-2). In the chemical formulae (R6-1) and (R6-2) below, m, n, o, p, q, r, and s are positive integers, are not particularly limited, and are, for example, 1 to 10, 1 to 5, or 1 to 3. Specific examples of the substituent containing the heterocycle include groups represented by the following chemical formulae (R6-1-2) and (R6-2-2). One or more of the hydrogen atoms bonded to the carbon atoms of the alkyl group, the hydroxyalkyl group, the cycloalkyl group, and the substituent containing the heteroatom may be substituted with another substituent. Examples of such a substituent are as described above.

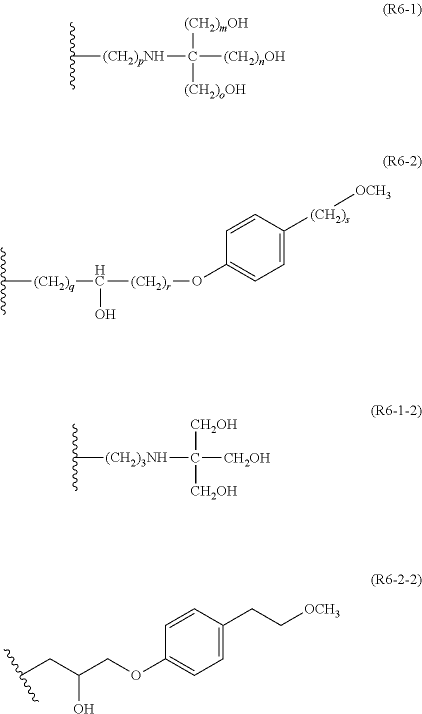

In the chemical formula (1), R¹ and R⁶ may together form a ring structure. The carbon number of the ring structure is not particularly limited, and may be, for example, 1 to 40, 1 to 32, 1 to 24, 1 to 18, 1 to 12, or 1 to 6. The ring structure may or may not have, for example, a heteroatom. The heteroatom is, for example, as described above. Specifically, the ring structure can be, for example, a structure represented by the following chemical formula (R1R6). In the following chemical formula (R1R6), R², R³, R⁴, and R⁵ are as described above, for example. One or more of the hydrogen atoms bonded to the carbon atoms of the ring structure may be substituted with another substituent. Examples of such a substituent are as described above.

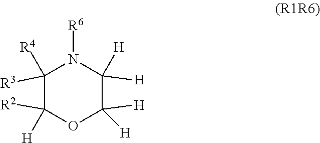

In the chemical formula (1), R⁵ and R⁶ may together form a ring structure. The carbon number of the ring structure is not particularly limited, and may be, for example, 1 to 40, 1 to 32, 1 to 24, 1 to 18, 1 to 12, or 1 to 6. The ring structure may or may not have, for example, a heteroatom. The heteroatom is, for example, as described above. Specifically, the ring structure can be, for example, a structure represented by the following chemical formula (R5R6). In the following chemical formula (R5R6), R¹, R², R³, and R⁴ are as described above, for example. In following chemical formula (R5R6), t is a positive integer and is not particularly limited, and is, for example, 1 to 10, 1 to 5, 1 to 3, or 2. Specifically, the ring structure can be, for example, a structure represented by the following chemical formula (R5R6-2). In the following chemical formula (R5R6-2), R¹, R², R³, and R⁴ are as described above, for example. One or more of the hydrogen atoms bonded to the carbon atoms of the ring structure may be substituted with another substituent. Examples of such a substituent are as described above.

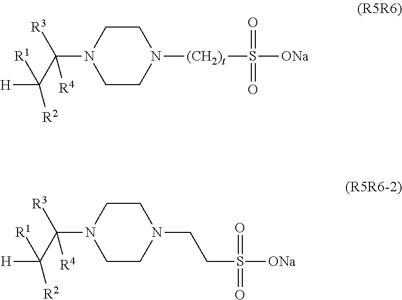

Specific examples of the compound represented by the chemical formula (1) include 2-amino-1-cyclohexylethanol, 2-aminoethanol, 1-amino-2-propanol, 2-amino-1-phenylethanol, 1,3-bis[tris(hydroxymethyl)methylamino]propane, N-cyclohexylethanolamine, diethanolamine, diethylamine, dipropylamine, sodium HEPES salt, methopropyl tartrate, morpholine, octopamine, propylamine, triethanolamine, triethylamine, timolol maleate, and trishydroxymethylaminomethane.

The table 1 below shows the structures of the drugs of the present invention by combinations of R¹, R², R³, R⁴, R⁵, and R⁶ in the chemical formula (1). In the table 1, “R6-1-2” and “R6-2-2” represent groups represented by the chemical formula (R6-1-2) and the chemical formula (R6-2-2), respectively. “(R5R6-2)” indicates that R⁵ and R⁶ in the chemical formula (1) together form a ring structure represented by the chemical formula (R5R6-2). In addition, “(R1R6)” indicates that R¹ and R⁶ in the chemical formula (1) together form a ring structure represented by the chemical formula (R1R6).

TABLE 1A

| | Substituent | | | | | |
|---|---|---|---|---|---|---|
| Compound name | R¹ | R² | R³ | R⁴ | R⁵ | R⁶ |
| 2-amino-1-cyclohexylethanol | Hydroxy group | Cyclohexyl group | Hydrogen atom | Hydrogen atom | Hydrogen atom | Hydrogen atom |
| 2-aminoethanol | Hydroxy group | Hydrogen atom | Hydrogen atom | Hydrogen atom | Hydrogen atom | Hydrogen atom |
| 1-amino-2-propanol | Hydroxy group | Methyl group | Hydrogen atom | Hydrogen atom | Hydrogen atom | Hydrogen atom |
| 2-amino-1-phenylethanol | Hydroxy group | Phenyl group | Hydrogen atom | Hydrogen atom | Hydrogen atom | Hydrogen atom |
| 1,3-bis[tris(hydroxy- | Hydroxy group | Hydrogen atom | Hydroxymethyl | Hydroxymethyl | Hydrogen atom | Chemical |
| methyl)methylamino]propane | | | group | group | | formula: R6-1-2 |
| N-cyclohexylethanolamine | Hydroxy group | Hydrogen atom | Hydrogen atom | Hydrogen atom | Hydrogen atom | Cyclohexyl group |
| Diethanolamine | Hydroxy group | Hydrogen atom | Hydrogen atom | Hydrogen atom | Hydrogen atom | 2-hydroxyethyl |
| | | | | | | group |
| Diethylamine | Hydrogen atom | Hydrogen atom | Hydrogen atom | Hydrogen atom | Hydrogen atom | Ethyl group |
| Dipropylamine | Hydrogen atom | Methyl group | Hydrogen atom | Hydrogen atom | Hydrogen atom | Propyl group |
| Sodium HEPES salt | Hydroxy group | Hydrogen atom | Hydrogen atom | Hydrogen atom | (Chemical formula: R5R6-2) | |
| Methopropyl tartrate | Hydrogen atom | Hydrogen atom | Hydrogen atom | Methyl group | Hydrogen atom | Chemical |
| | | | | | | formula: R6-2-2 |
| Morpholine | (Chemical | Hydrogen atom | Hydrogen atom | Hydrogen atom | Hydrogen atom | (Chemical |
| | formula: R1R6) | | | | | formula: R1R6) |
| Octopamine | Hydroxy group | 4-hydroxyphenyl | Hydrogen atom | Hydrogen atom | Hydrogen atom | Hydrogen atom |
| | | group | | | | |

TABLE 1B

| | Substituent | | | | | |
|---|---|---|---|---|---|---|
| Compound name | R¹ | R² | R³ | R⁴ | R⁵ | R⁶ |
| Propylamine | Hydrogen atom | Methyl group | Hydrogen atom | Hydrogen atom | Hydrogen atom | Hydrogen atom |
| Triethanolamine | Hydroxy group | Hydrogen atom | Hydrogen atom | Hydrogen atom | 2-hydroxyethyl | 2-hydroxyethyl |
| | | | | | group | group |
| Triethylamine | Hydrogen atom | Hydrogen atom | Hydrogen atom | Hydrogen atom | Ethyl group | Ethyl group |
| Timolol maleate | Hydroxy group | Chemical formula: R2 | Hydrogen atom | Hydrogen atom | Hydrogen atom | Tert-butyl group |
| Trishydroxymethyl- | Hydroxy group | Hydrogen atom | Hydroxymethyl | Hydroxymethyl | Hydrogen atom | Hydrogen atom |
| aminomethane | | | group | group | | |

Each of the above-described drugs is a known drug. The inventors of the present invention have found that these drugs, which are the compounds represented by the chemical formula (1), accelerate glucose consumption and glycolysis, although the mechanism is unknown, thereby made the present invention.

In the present invention, the “glucose consumption” may be, for example, acceleration of a decrease in the glucose concentration or suppression of an increase in the glucose concentration. The “glucose consumption” may be measured, for example, by the method described in the examples below. The “glucose consumption” may be, for example, consumption of glucose by cells. In this case, the “glucose consumption” can also be referred to as, for example, “uptake of glucose into cells”. Thus, the glucose consumption accelerator of the present invention can also be referred to as, for example, an uptake accelerator of glucose into cells. It is known that glucose is taken up, for example, via a glucose transporter present in a cell membrane. Thus, the glucose consumption accelerator of the present invention can also be referred to as, for example, an activator of a signaling cascade via a glucose transporter. However, the present invention is not limited thereto.

In the present invention, the “glycolysis” is, for example, a metabolic system starting from glucose. A metabolite of the glycolysis can be, for example, a lactate. Therefore, for example, as described below, by measuring the concentration of the lactate, the acceleration of the glycolysis can be examined. The glycolysis is also referred to as an anaerobic glycolysis, for example.

According to the glucose consumption accelerator of the present invention, as described above, the consumption of glucose can be accelerated. According to the glycolysis accelerator of the present invention, the glycolysis can be accelerated as described above. Thus, the glucose consumption accelerator and the glycolysis accelerator of the present invention can be used as pharmaceutical compositions for use in the treatment of a disease caused by glucose concentration in vivo, for example. The disease can be, for example, diabetes. In the present invention, “treatment” includes, for example, the meaning of prevention of the disease, the amelioration of the disease, and the amelioration of the prognosis of the disease, and may be any of them.

According to the glucose consumption accelerator of the present invention, for example, it is possible to accelerate the consumption of glucose and accelerate the glycolysis without suppressing the metabolism of lactate produced by the consumption of glucose.

The glucose consumption accelerator of the present invention may contain, for example, only one type of the drugs of the present invention or two or more types of them in combination as an active ingredient(s), and the number of types of the drugs to be contained is not particularly limited. The glucose consumption accelerator of the present invention includes the drug of the present invention, for example, as a main ingredient.

The glucose consumption accelerator of the present invention may be used, for example, in vivo or in vitro. The glucose consumption accelerator of the present invention can be used, for example, as a research reagent or, as described above, as a pharmaceutical.

The subject to be administered is not particularly limited. When the glucose consumption accelerator of the present invention is used in vivo, the subject to be administered is not particularly limited, and is, for example, a human or a non-human animal excluding a human. Examples of the non-human mammals include non-human animals such as mice, rats, rabbits, dogs, sheep, horses, cats, goats, monkeys, and guinea pigs. When the glucose consumption accelerator of the present invention is used in vitro, examples of the subject to be administered include cells, tissues, and organs. The cell may be, for example, cells collected from a living body, cultured cells, and the like. The cell is not particularly limited and examples thereof include fibroblasts, hepatocytes, and adipocytes.

The conditions of use of the glucose consumption accelerator of the present invention (hereinafter also referred to as “administration conditions”) are not particularly limited, and for example, the administration form, administration timing, dosage, and the like can be appropriately determined according to the type or the like of the subject to be administered. When the glucose consumption accelerator of the present invention is used in vivo, examples of the administration form include oral administration, intraperitoneal administration, and subcutaneous administration.

The dosage form of the glucose consumption accelerator of the present invention is not particularly limited, and can be appropriately determined according to the administration form, for example. In the case of oral administration, examples of the dosage form include capsules, extracts, elixirs, granules, pills, suspensions, fine granules, powders, spirits, tablets, syrups, dips and decoctions, tinctures, aromatics, lemonades, and stream extracts.

The glucose consumption accelerator of the present invention may contain, for example, an additive as required. When the glucose consumption accelerator of the present invention is used as a medicament, the additive is preferably a pharmaceutically acceptable additive. Examples of the additive include excipients, stabilizers, preservatives, buffers, corrigents, suspending agents, emulsifying agents, flavoring agents, dissolution assists, coloring agents, and viscosifiers. The additive may be, for example, an oral additive. Examples of the oral additive include caries preventive agents, intestinal regulators, and corrigents. In the present invention, the blending amount of the additive is not particularly limited, and it is only required not to hinder the function of the glucose consumption accelerator.

The conditions for administrating the glucose consumption accelerator of the present invention are not particularly limited, and, for example, the administration timing, administration period, dosage, and the like can be appropriately determined according to the type, sex, age, site of the subject to be administered, and the like.

As a specific example, when the glucose consumption accelerator of the present invention is orally administered to a human, the total dosage per day is, for example, 100 to 5000 mg or 500 to 2500 mg. The number of times of administration per day is, for example, 1 to 5 times or 2 to 3 times.

(Pharmaceutical Composition)

The pharmaceutical composition for use in the treatment of diabetes according to the present invention is characterized in that it includes the glucose consumption accelerator or the glycolysis accelerator of the present invention, as described above. The pharmaceutical composition of the present invention is characterized in that it includes the glucose consumption accelerator of the present invention or the glycolysis accelerator, and other constitution and conditions are not particularly limited. Regarding the pharmaceutical composition of the present invention, reference can be made to the description as to the glucose consumption accelerator and the glycolysis accelerator of the present invention. According to the pharmaceutical composition of the present invention, for example, diabetes can be treated.

(Glucose Consumption Accelerating Method and Glycolysis Accelerating Method)

The glucose consumption accelerating method of the present invention is characterized in that it includes a step of administering the glucose consumption accelerator of the present invention to a subject to be administered. Also, the glycolysis accelerating method of the present invention is characterized in that it includes a step of administering the glycolysis accelerator of the present invention to a subject to be administered. The present invention is characterized by including the step of administering the glucose consumption accelerator or glycolysis accelerator of the present invention, and other steps and conditions are not particularly limited. The glucose consumption accelerator or glycolysis accelerator of the present invention is as described above. The conditions for administrating the glucose consumption accelerator and glycolysis accelerator of the present invention are not particularly limited, and, for example, are the same as those described in the description as to the glucose consumption accelerator of the present invention.

(Use of Drug)

The present invention is the use of the drug of the present invention for use in accelerating glucose consumption and glycolysis, and the use of the drug for use in the treatment of diabetes. The present invention is the use of the drug for producing the glucose consumption accelerator and glycolysis accelerator, and the use of the drug for producing a pharmaceutical composition for use in the treatment of diabetes. Regarding this invention, for example, reference can be made to the descriptions as to the glucose consumption accelerator and glycolysis accelerator, the pharmaceutical composition, the glucose consumption accelerating method, and glycolysis accelerating method of the present invention.

### EXAMPLES

The examples of the present invention are described below. The present invention, however, is not limited by the following examples. The commercially available reagents were used based on the protocols thereof, unless otherwise specified.

### Example 1

The present example examined that the drug of the present invention has a glucose consumption accelerating effect on fibroblasts.

As drugs, 2-amino-1-cyclohexylethanol (Matrix Biochemicals), 2-aminoethanol (Tokyo Chemical Industry Co., Ltd.), 1-amino-2-propanol (Wako Pure Chemical Industries, Ltd.), 2-amino-1-phenylethanol (Tokyo Chemical Industry Co., Ltd.), 1, 3-bis[tris(hydroxymethyl)-methylamino]propane (Tokyo Chemical Industry Co., Ltd.), N-cyclohexylethanolamine (Tokyo Chemical Industry Co., Ltd.), diethanolamine (Wako Pure Chemical Industries, Ltd.), diethylamine (Wako Pure Chemical Industries, Ltd.), dipropylamine (Tokyo Chemical Industry Co., Ltd.), HEPES sodium salt (MP Biomedicals), metoprolol tartrate (Tokyo Chemical Industry Co., Ltd.), morpholine (Wako Pure Chemical Co., Ltd.), octopamine (MP Biomedicals), propylamine (Tokyo Chemical Industry Co., Ltd.), triethylamine (Wako Pure Chemical Co., Ltd.), timolol maleate (Wako Pure Chemical Industries, Ltd.) and tris(hydroxymethyl)aminomethane (Wako Pure Chemical Industries, Ltd.) were used. Each drug was dissolved in distilled water to prepare a sample.

Rat-derived fibroblasts (Py-3Y1-S2, subculture strain) were then seeded in a 24-well microplate to achieve a density of 2×10⁵ cells/ml/well and cultured up to be monolayers. In the culture, DMEM (Nissui Pharmaceutical Co., Ltd.) was used as a culture medium. To this culture solution, the samples were added so that each drug had a final concentration of 0.5 mg/ml, and further cultured for 12 to 24 hours. As a control of the drug, the culture was performed in the same manner except that distilled water to which the drug was not added was added to the culture solution instead of the sample.

After the addition of the drug, the glucose concentration of each of the culture solutions was measured immediately after the start of the culture and after the completion of the culture. The glucose concentration was measured using a glucose assay kit (Waco). Then, the glucose concentration after the completion of the culture was divided by the glucose concentration immediately after the start of the culture (5.6 mmol per liter) to calculate the glucose consumption. Further, the glucose consumption in the control was set as a reference value of 100, and the relative value of the glucose consumption in each sample was calculated.

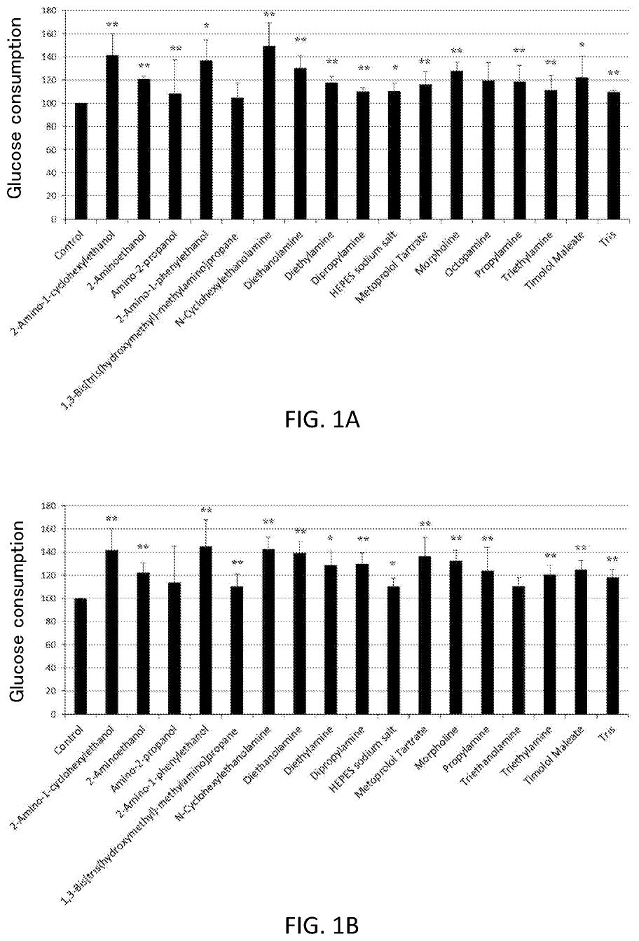

The results are shown in FIG. 1A. FIG. 1A is a graph showing the relative values of the glucose consumption of fibroblasts to which the above-described drugs each having a concentration of 0.5 mg/ml were added. In FIG. 1A, the vertical axis represents the relative value of glucose consumption, and the horizontal axis represents the drug. As shown in FIG. 1A, in every sample, the addition of each drug resulted in a relative glucose consumption value of 100 or more, and an increase in the glucose consumption. Among them, the addition of 2-amino-1-cyclohexylethanol, 2-aminoethanol, 1-amino-2-propanol, 2-amino-1-phenylethanol, N-cyclohexylethanolamine, diethanolamine, diethylamine, dipropylamine, HEPES sodium salt, metoprolol tartrate, morpholine, propylamine, triethylamine, timolol maleate, and tris(hydroxymethyl)aminomethane significantly increased glucose consumption compared to a control (t-test: p<0.05 or p<0.1, indicated by “**” or “*” in FIG. 1A), which showed strong glucose consumption accelerating effect.

Furthermore, the glucose consumption accelerating effect was examined under the same conditions as described above except that 2-amino-1-cyclohexylethanol, 2-aminoethanol, 1-amino-2-propanol, 2-amino-1-phenylethanol, 1, 3-bis[tris(hydroxymethyl)-methylamino]propane, N-cyclohexylethanolamine, diethanolamine, diethylamine, dipropylamine, HEPES sodium salt, metoprolol tartrate, morpholine, propylamine, triethanolamine (Tokyo Chemical Industry Co., Ltd.), triethylamine, timolol maleate, and tris(hydroxymethyl)aminomethane were used as drugs and each drug had a final concentration of 1 mg/ml.

The results are shown in FIG. 1B. FIG. 1B is a graph showing the relative values of the glucose consumption of fibroblasts to which the above-described drugs each having a concentration of 1 mg/ml were added. In FIG. 1B, the vertical axis represents the relative value of glucose consumption, and the horizontal axis represents the drug. As shown in FIG. 1B, in every sample, the addition of each drug resulted in a relative glucose consumption value of 100 or more, and an increase in the glucose consumption. Among them, the addition of 2-Amino-1-cyclohexylethanol, 2-Aminoethanol, 2-Amino-1-phenylethanol, 1, 3-Bis[tris(hydroxymethyl)-methylamino]propane, N-Cyclohexylethanolamine, Diethanolamine, Diethylamine, Dipropylamine, HEPES sodium salt, Metoprolol Tartrate, Morpholine, Propylamine, Triethylamine, Timolol maleate, and tris(hydroxymethyl)aminomethane significantly increased glucose consumption compared to a control (t-test: p<0.05 or p<0.1, indicated by “**” or “*” in FIG. 1), which showed strong glucose consumption accelerating effect.

Next, as a comparative example, the glucose consumption accelerating effect was examined under the same conditions as described above except that tricine and trimethylolpropane, which are drugs having no structures represented by the chemical formula (1), were used.

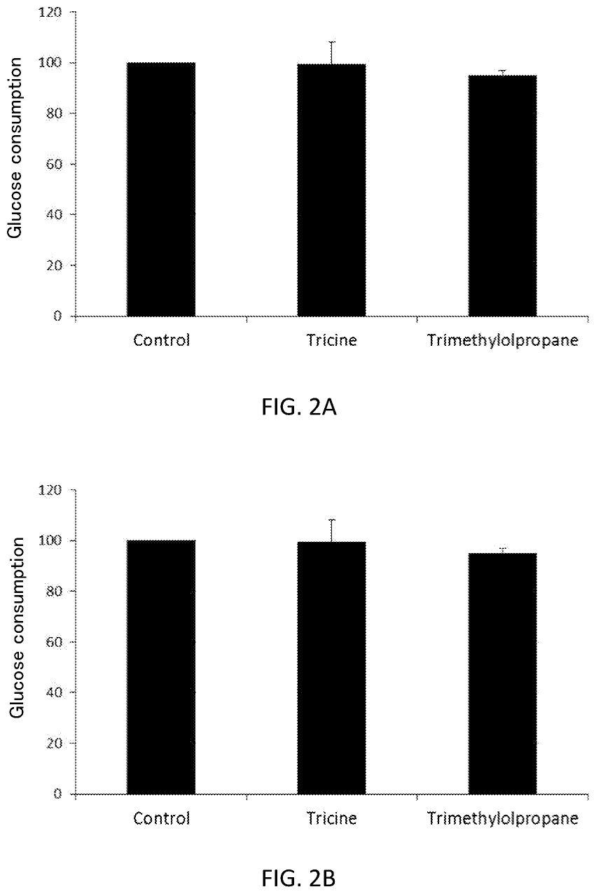

The results are shown in FIGS. 2A and 2B. FIGS. 2A and 2B are graphs each showing the relative values of the glucose consumption of fibroblasts to which the above-described drugs of the comparative example were added. FIG. 2A shows the result of the case in which each drug of the comparative example had a concentration of 0.5 mg/ml, and FIG. 1B shows the result of the case in which each drug of the comparative example had a concentration of 1 mg/ml. In FIGS. 2A and 2B, the vertical axis represents the relative value of the glucose consumption, and the horizontal axis represents the drug. As shown in FIGS. 2A and 2B, the addition of each drug of the comparative example having a concentration of 0.5 mg/ml or 1 mg/ml resulted in a relative glucose consumption value of less than 100 and no increase in glucose consumption in any of the samples.

The results shown in FIG. 1A to FIG. 2B showed that the drugs of the present invention exhibited the glucose consumption accelerating effect by having the structure represented by the chemical formula (1).

### Example 2

The present example examined that the drug of the present invention has a glucose consumption accelerating effect on fibroblasts in a dose dependent manner.

Samples of 2-amino-1-cyclohexylethanol and 2-amino-1-phenylethanol, each of which exhibited a strong glucose consumption accelerating effect in Example 1, were prepared. The fibroblasts were cultured under the same conditions as in Example 1. To this culture solution, the samples were added so that each drug had final concentrations of 0.1, 0.25, and 0.5 mg/ml. The relative value of the glucose consumption in each sample was calculated in the same manner as in Example 1.

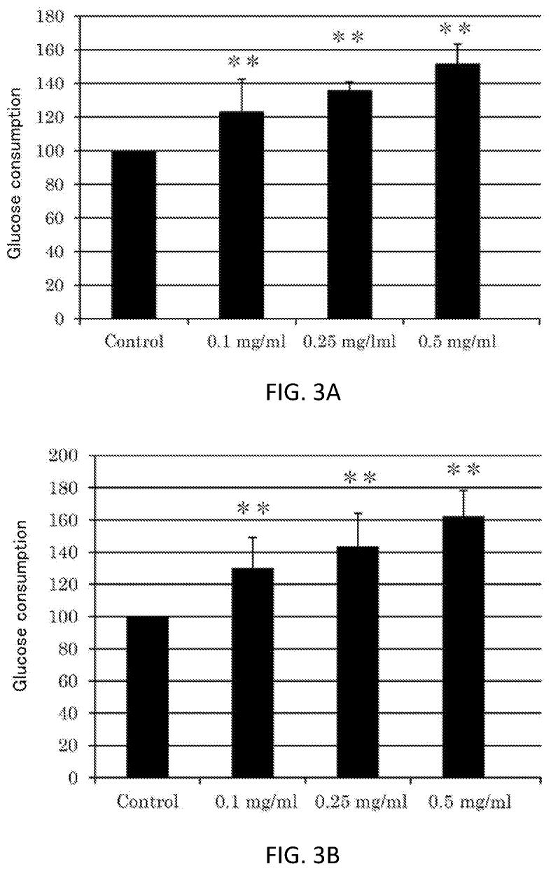

The results are shown in FIGS. 3A and 3B. FIGS. 3A and 3B are graphs each showing the relative values of the glucose consumption of fibroblasts to which the above-described drugs were added. FIG. 3A shows the result of the case of adding 2-amino-1-cyclohexylethanol, and FIG. 3B shows the result of the case of adding 2-amino-1-phenylethanol. In FIGS. 3A and 3B, the vertical axis represents the relative value of the glucose consumption, and the horizontal axis represents the concentration of each drug. As shown in FIG. 3A, the addition of 0.1, 0.25, and 0.5 mg/ml 2-amino-1-cyclohexylethanol to fibroblasts increased glucose consumption in a dose-dependent manner up to about 120, about 130, and about 150, respectively. The glucose concentration was significantly increased compared to the control (t-test: p<0.05, indicated by “**” in FIGS. 3A and 3B) at any concentration, which showed a strong glucose consumption accelerating effect. Furthermore, as shown in FIG. 3B, the addition of 0.1, 0.25, and 0.5 mg/ml 2-amino-1-phenylethanol increased glucose consumption in a dose-dependent manner up to about 130, about 140, and about 160, respectively. The glucose concentration was significantly increased compared to the control, which showed a strong glucose consumption accelerating effect.

As shown in each graph of FIGS. 3A and 3B, all of the administered drugs exhibited glucose consumption accelerating effects in a dose dependent manner. In addition, it was found that the glucose consumption accelerating effect was effectively obtained even at a low dose.

### Example 3

The present example examined that the drug of the present invention has a glucose consumption accelerating effect on fibroblasts in which glucose consumption was suppressed.

First, samples of streptozotocin (STZ), alloxan, and nicotinamide were prepared in the same manner as in Example 1. The fibroblasts were cultured. To this culture solution, the samples were added so that each drug had a final concentration 1 mg/ml. Streptozotocin, alloxan, and nicotinamide are drugs all known to produce symptoms of diabetes when administered to the body. The relative value of the glucose consumption was calculated in the same manner as in Example 1.

Next, samples of streptozotocin, alloxan, and nicotinamide plus 2-amino-1-phenylethanol (2-A-1-P) were prepared in the same manner. The concentration of 2-A-1-P was 1 mg/ml. The relative value of the glucose consumption was calculated in the same manner.

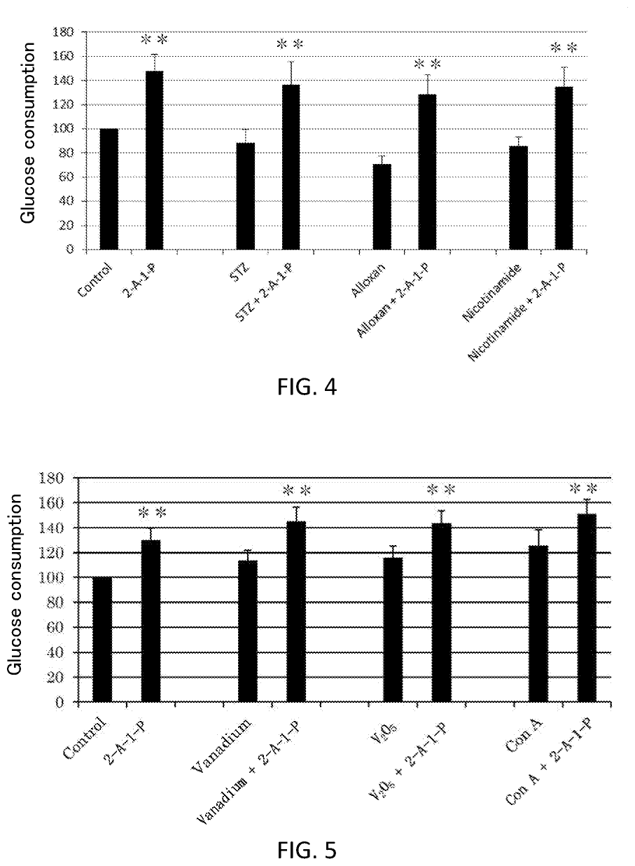

The results are shown in FIG. 4. FIG. 4 is a graph showing the relative values of the glucose consumption of fibroblasts to which the above-described drugs were added. In FIG. 4, the vertical axis represents the relative value of the glucose consumption, and the horizontal axis represents the drug. As shown in FIG. 4, the addition of 1 mg/ml streptozotocin, alloxan, and nicotinamide resulted in glucose consumption relative values of about 90, about 70, and about 85, respectively, and a decrease in glucose consumption. On the other hand, the addition of 2-A-1-P in addition to streptozotocin, alloxan, and nicotinamide resulted in a glucose consumption relative value of 100 or more, and recovery of the glucose consumption. This showed that 2-amino-1-phenylethanol exhibited the glucose consumption accelerating effect also on fibroblasts in which glucose consumption was suppressed.

### Example 4

The present example examined that the drug of the present invention has further glucose consumption accelerating effect on fibroblasts in which glucose consumption was accelerated.

First, samples of vanadium, V₂O₅, and concanavalin A (ConA) were prepared in the same manner as in Example 1. The fibroblasts were cultured. To this culture solution, the samples were added so that vanadium had a final concentration of 1.0 mg/ml and ConA had a final concentration of 100 μg/ml. Vanadium, V₂O₅, and concanavalin A are drugs all known to exhibit insulin-like effects upon administration to fibroblasts. The relative value of the glucose consumption in each sample was calculated in the same manner as in Example 1.

Next, samples of vanadium, V₂O₅, and concanavalin A plus 2-amino-1-phenylethanol (2-A-1-P) were prepared in the same manner. The concentration of 2-A-1-P was 0.5 mg/ml. The relative value of the glucose consumption was calculated in the same manner.

The results are shown in FIG. 5. FIG. 5 is a graph showing the relative values of the glucose consumption of fibroblasts to which the above-described drugs were added. In FIG. 5, the vertical axis represents the relative value of the glucose consumption, and the horizontal axis represents the drug. As shown in FIG. 5, the addition of vanadium, V₂O₅, and concanavalin A resulted in glucose consumption relative values of about 110, about 115, and about 120, respectively, and an increase in glucose consumption. On the other hand, the addition of 2-A-1-P in addition to vanadium, V₂O₅ and concanavalin A resulted in glucose consumption relative values of about 145, about 140, and about 150, respectively, and a further increase in the glucose consumption. This showed that 2-amino-1-phenylethanol exhibited the glucose consumption accelerating effect also on fibroblasts in which glucose consumption was accelerated by vanadium, V₂O₅ and concanavalin A.

### Example 5

The present example examined that the drug of the present invention has a glucose consumption accelerating effect on liver cancer cells.

First, samples of 2-amino-1-cyclohexylethanol and 2-amino-1-phenylethanol were prepared in the same manner as in Example 1. Instead of the rat-derived fibroblasts (Py-3Y1-S2, subculture strain), rat-derived liver cancer cells (Ry121B, subculture line) were used, and the rat-derived liver cancer cells were cultured under the same conditions as in Example 1. To this culture solution, the samples were added so that each drug had a final concentration of 1 mg/ml. As a control, the culture was performed in the same manner except that distilled water to which the drug was not added was added to the culture solution instead of the sample. The relative value of the glucose consumption in each sample was calculated in the same manner as in Example 1.

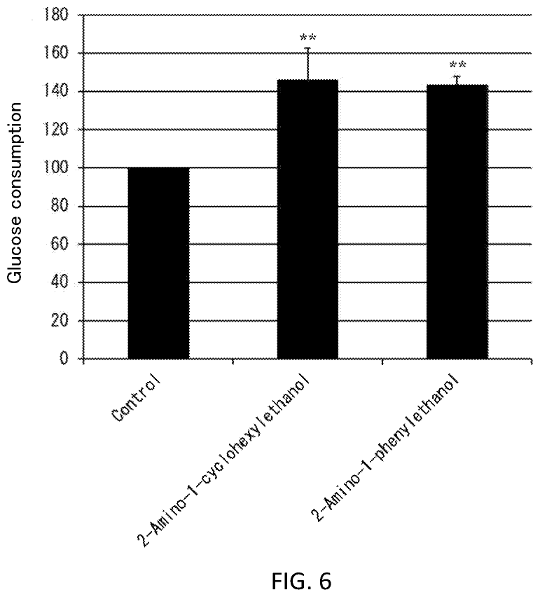

The results are shown in FIG. 6. FIG. 6 is a graph showing the relative values of the glucose consumption of liver cancer cells to which the above-described drugs were added. In FIG. 6, the vertical axis represents the relative value of the glucose consumption, and the horizontal axis represents the drug.

As shown in FIG. 6, also in liver cancer cells, the addition of 2-amino-1-cyclohexylethanol and 2-amino-1-phenylethanol also resulted in glucose consumption relative values of about 145 and about 140, respectively, and a significant increase in glucose consumption compared to the control.

The results shown in FIG. 6 showed that the drug of the present invention exhibited the glucose consumption accelerating effect also on liver cancer cells.

### Example 6

The present example examined that the glucose consumption accelerating effect by the drug of the present invention accelerates the glycolysis by cells.

First, samples of 2-amino-1-cyclohexylethanol and 2-amino-1-phenylethanol were prepared in the same manner as in Example 1. The fibroblasts and the liver cancer cells were cultured under the same conditions as in Examples 1 and 5. To these culture solutions, the samples were added so that each drug had a final concentration of 1 mg/ml. As a control, the culture was performed in the same manner except that distilled water to which the drug was not added was added to the culture solution instead of the sample.

After the addition of the drug, the culture solution was diluted so that the weight ratio of the culture solution to distilled water was 1:19, and the lactate concentration of the diluted culture solution was measured. The lactate concentration was measured using a lactate assay kit (product name: Lactate Assay Kit-WST, product of Dojindo Laboratories). The lactate concentration in the control was set as a reference value of 100, and the relative value of the lactate concentration in each sample was calculated.

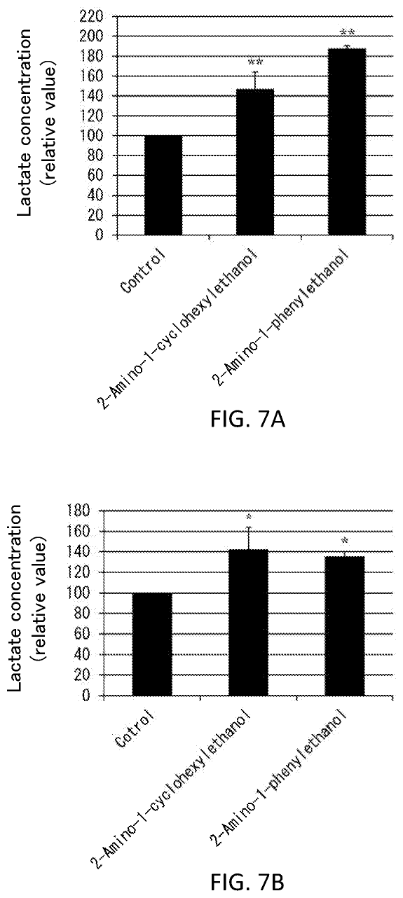

The results are shown in FIGS. 7A and 7B. FIGS. 7A and 7B are graphs each showing the relative values of the lactate concentration of the cells to which the drugs were added. FIG. 7A shows the results of fibroblasts, and FIG. 7B shows the results of liver cancer cells. In FIGS. 7A and 7B, the vertical axis represents the relative value of the lactate concentration, and the horizontal axis represents the drug.

As shown in FIG. 7A, in fibroblasts, the addition of 2-amino-1-cyclohexylethanol and 2-amino-1-phenylethanol increased the lactate concentration up to about 145 and about 185, respectively. As shown in FIG. 7B, in liver cancer cells, the addition of 2-amino-1-cyclohexylethanol and 2-amino-1-phenylethanol increased the lactate concentration up to about 140 and about 135, respectively.

The results shown in FIGS. 7A and 7B showed that the drug of the present invention increased the lactate concentration in fibroblasts and liver cancer cells, and exhibited the glucose consumption accelerating effect.

### Example 7

The present example examined that the drug of the present invention lowers the blood glucose level of mice.

As a drug, 2-amino-1-phenylethanol was used. First, glucose (KANTO CHEMICAL CO., INC.) was dissolved in water for injection so as to achieve a concentration of 200 mg/ml, thereby preparing a glucose solution. Next, the drug was dissolved in the water for injection, and then the glucose solution was added thereto so that the drug and glucose had final concentrations of 2.5 mg/ml and 100 mg/ml, respectively, thereby preparing a sample. As a control of the drug, the culture was performed in the same manner except that 100 mg/ml glucose solution to which the drug was not added was used instead of the sample.

Male mice of 6-week-old ICR strain were purchased from Japan SLC, Inc., and preliminary rearing was carried out for about 1 week to check there was no abnormality in performance status. The conditions for the preliminary rearing were as follows: 4 mice each were housed in a polycarbonate cage, room temperature was 23° C.±3° C., and illumination time was 12 hours/day. Diets (mice and rat chow, product of Nosan Corporation) and drinking water (tap water) were freely ingested.

After the preliminary rearing, the mice were fasted for about 21 hours, and then body weight and blood glucose levels were measured. The mice were divided into the total of two groups, namely, a test group and a control group to ensure that there was no variation in blood glucose levels between the groups. The number of animals in each group was eight. The table 2 shows the body weight (g) of the mice at the time of grouping.

TABLE 2

| Group | Test animal | Body weight (g) |
|---|---|---|
| Control group | No. 1 | 30.9 |
| | No. 2 | 29.6 |
| | No. 3 | 29.9 |
| | No. 4 | 31.0 |
| | No. 5 | 27.7 |
| | No. 6 | 31.4 |
| | No. 7 | 31.5 |
| | No. 8 | 29.6 |
| Test group | No. 1 | 29.2 |
| | No. 2 | 32.3 |
| | No. 3 | 30.8 |
| | No. 4 | 30.2 |
| | No. 5 | 28.4 |
| | No. 6 | 28.0 |
| | No. 7 | 29.4 |
| | No. 8 | 30.4 |

After the grouping, mice of the test group and the control group were orally administered a single dose of the sample and the control, respectively, using a gastric sonde to a dose volume of 20 ml/kg. That is, the test group mice were administered the drug and glucose at doses of 50 mg/kg and 2000 mg/kg, respectively, and the control group mice were administered glucose at a dose of 2000 mg/kg. With the time of the administration being regarded as 0 minutes, the blood glucose level was measured at 30 minutes, 60 minutes, 90 minutes, and 120 minutes. The blood glucose level was measured by using an ACCU-CHEK Aviva (Roche Diagnostics K.K.), blood was collected by inserting an injection needle into the tip of the tail, and the blood glucose level was measured for the collected blood.

As to the blood glucose levels at 0 minutes, 30 minutes, 60 minutes, 90 minutes and 120 minutes, the test group and the control group were compared by t-test. The significance levels were 5% and 1%.

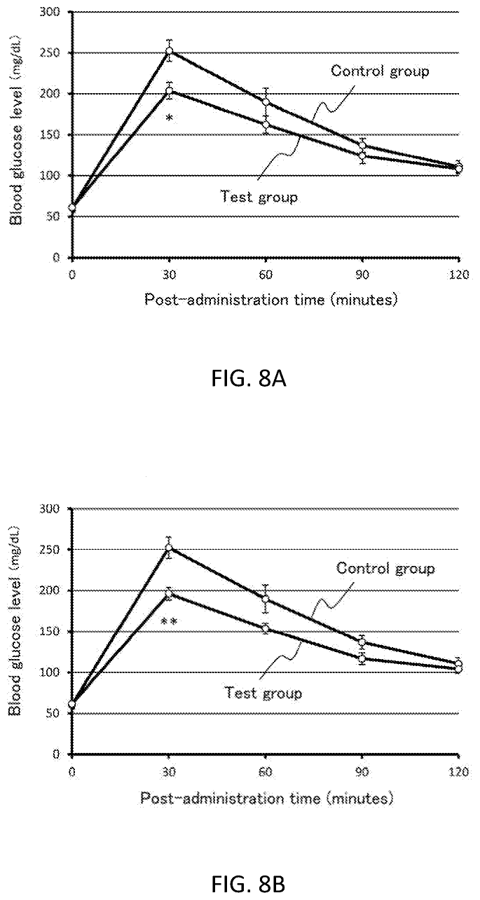

The results are shown in the table 3 and FIGS. 8A and 8B. The table 3 shows the measured values (mg/dL) of the blood glucose levels in the individuals at the predetermined time after the administration, and FIGS. 8A and 8B are graphs each showing the mean values of the blood glucose levels in the test group and the control group at the predetermined time after the administration. In FIGS. 8A and 8B, the vertical axis represents the blood glucose level (mg/dL), and the horizontal axis represents the post-administration time (minutes). In FIG. 8A, the mean value was calculated based on the blood glucose levels obtained for all the individuals (No. 1 to No. 8) in the control group and the test group. On the other hand, as shown in the table 3, the blood glucose level after glucose administration of one individual (No. 8) in the test group showed abnormal values compared to the calculated values of the other seven individuals (No. 1 to No. 7) in the test group. Therefore, in FIG. 8B, the data of one individual (No. 8) in the test group showing the abnormal values was excluded, and the mean value was calculated based on the blood glucose levels obtained for the other seven individuals (No. 1 to No. 7) in the test group.

TABLE 3

| | Test | Post-administration time (minutes) | | | | |
|---|---|---|---|---|---|---|
| Group | animal | 0 | 30 | 60 | 90 | 120 |
| Control | No. 1 | 41 | 237 | 166 | 123 | 90 |
| group | No. 2 | 47 | 294 | 231 | 138 | 107 |
| | No. 3 | 55 | 193 | 147 | 123 | 92 |
| | No. 4 | 53 | 268 | 268 | 189 | 149 |
| | No. 5 | 81 | 309 | 221 | 144 | 134 |
| | No. 6 | 67 | 251 | 155 | 125 | 100 |
| | No. 7 | 81 | 233 | 130 | 114 | 104 |
| | No. 8 | 60 | 235 | 201 | 140 | 110 |
| Test group | No. 1 | 46 | 177 | 151 | 121 | 116 |
| | No. 2 | 53 | 194 | 136 | 93 | 91 |
| | No. 3 | 61 | 194 | 180 | 138 | 112 |
| | No. 4 | 70 | 222 | 171 | 138 | 113 |
| | No. 5 | 54 | 165 | 136 | 117 | 108 |
| | No. 6 | 60 | 202 | 141 | 91 | 79 |
| | No. 7 | 86 | 219 | 160 | 120 | 111 |
| | No. 8 | 59 | 256 | 223 | 176 | 134 |

As shown in FIGS. 8A and 8B, the test group tended to have lower blood glucose levels at 30, 60, 90 and 120 minutes after administration compared to the control group. In particular, at 30 minutes after administration, the test group had significantly lower blood glucose levels (P<0.05, and P<0.01) compared to the control group.

In addition, the area under the curves (AUCs) were calculated for the measured blood glucose levels. The AUC was calculated by calculating the area surrounded by the measurement curve and a straight line parallel to the time axis and passing through the measured value at the time of administration in the graph with the measured value on the vertical axis and the time on the horizontal axis. Then, the test group and the control group were compared by t-test for AUC in the same manner as the comparison for the blood glucose level.

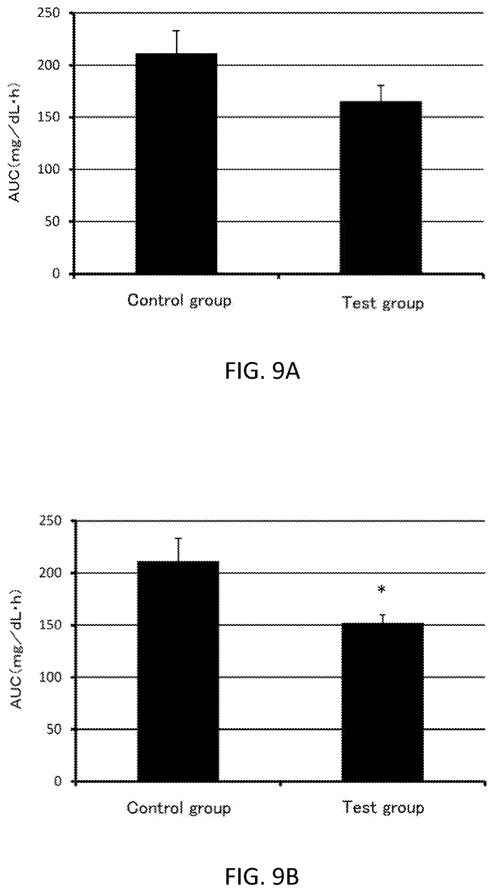

The results are shown in the table 4 and FIGS. 9A and 9B. The table 4 shows the calculated values of AUC (mg/dL·h) in the individuals, and FIGS. 9A and 9B are graphs each showing the mean values of AUC in the test group and the control group. In FIGS. 9A and 9B, the vertical axis represents AUC (mg/dL·h), and the horizontal axis represents the control group and the test group. In FIG. 9A, the mean value was calculated based on the calculated values of AUC obtained for all the individuals (No. 1 to No. 8) in the control group and the test group. On the other hand, as shown in Table 4, the calculated value (258) of AUC of one individual (No. 8) in the test group showed an abnormal value compared to the calculated values of the other seven individuals (No. 1 to No. 7) in the test group. Therefore, in FIG. 9B, the data of one individual (No. 8) in the test group showing the abnormal value was excluded, and the mean value was calculated based on the calculated values of AUC obtained for the other seven individuals (No. 1 to No. 7) in the test group.

TABLE 4

AUC

| | Test | Calculated | Mean value ± |
|---|---|---|---|
| Group | animal | value | Standard error |
| Control group | No. 1 | 214 | 211 ± 21 |
| | No. 2 | 276 | |
| | No. 3 | 158 | |
| | No. 4 | 307 | |
| | No. 5 | 229 | |
| | No. 6 | 173 | |
| | No. 7 | 123 | |
| | No. 8 | 211 | |
| Test group | No. 1 | 173 | 165 ± 15 |
| | No. 2 | 142 | |
| | No. 3 | 177 | |
| | No. 4 | 171 | |
| | No. 5 | 142 | |
| | No. 6 | 132 | |
| | No. 7 | 127 | |
| | No. 8 | 258 | |

As shown in FIG. 9A, the test group tended to have lower AUC values compared to the control group. As shown in FIG. 9B, the test group had significantly lower AUC value (P<0.05) compared to the control group.

The results shown in FIGS. 8A to 9B showed that the drug of the present invention lowered the blood glucose level of mice.

### Example 8

The present example examined that the drug of the present invention exhibits a lactate synthesis accelerating effect even after long-term culture.

First, a sample of 2-amino-1-cyclohexylethanol (2-amino-1-cyclohexyl EOH) was prepared in the same manner as in Example 1. In addition to the rat-derived fibroblasts (Py-3Y1-S2, subculture line), human-derived esophageal cancer cells (TE-13, subculture line), African green monkey-derived kidney epithelial cells (VERO, subculture line), and human-derived hepatocellular cancer cells (HepG2, subculture line) were used and cultured under the same conditions as in Example 1. To these culture solutions, the sample was added so that the drug had a final concentration of 0.5 mg/ml. Thereafter, the culture solutions were further cultured for 24 to 48 hours. The culture time after the addition of the sample was set to the same condition for each cell. As a control 1, the culture was performed in the same manner except that distilled water to which the drug was not added was added to the culture solution instead of the sample. As a control 2, a sample of biguanide was prepared in the same manner as in Example 1, and the culture was performed in the same manner as in Example 1 except that the sample of biguanide was added instead of the sample. Then, the lactate concentration in each sample was measured in the same manner as in Example 6. The total of five experiments (one for Py-3Y1-S2, TE-13 and HepG2 and two for VERO) were performed using the four types of cells.

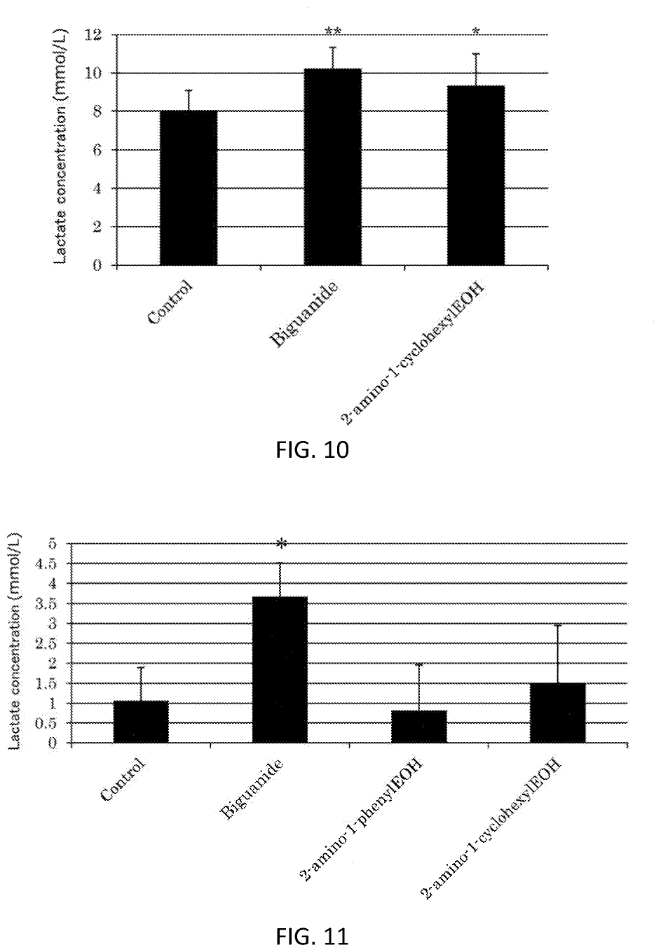

The results are shown in FIG. 10. FIG. 10 is a graph showing the lactate concentration in the culture solution of each cell to which the drugs were added. The value shown in the graph represents the mean value of the results of the five experiments. In FIG. 10, the vertical axis represents the lactate concentration (mmol/L) in the culture solution, and the horizontal axis represents the drug. As shown in FIG. 10, when 24 to 48 hours culture was performed after adding 0.5 mg/ml 2-amino-1-cyclohexylethanol, the lactate concentration was 9.35 mmol/L and the lactate concentration was increased compared to control 1 (Control) (P<0.05). Also in control 2 (Biguanide), the addition of biguanide increased the lactate concentration (P<0.01). This showed that 2-amino-1-cyclohexylethanol increased the lactate concentration even after long-term culture, and exhibited the glycolysis accelerating effect.

It seems that most of the glucose in the culture solution is changed to lactate after 24 to 48 hours culture. For example, since two molecules of lactate are synthesized from one molecule of glucose, if all of the glucose (5.6 mmol/L) immediately after the start of the culture is changed to lactate, the lactate concentration in the culture solution becomes 11.2 mmol/L. As shown in FIG. 10, when 24 to 48 hours culture was performed after adding each sample, the lactate concentration in the culture solution was about 8 to 10 mmol/L. This shows that, for example, about 70 to 90% of glucose was consumed and changed to lactate by culturing for 24 to 48 hours after addition of each sample. This also shows that 2-amino-1-cyclohexylethanol exhibits a glucose consumption accelerating effect even in the presence of, for example, a low concentration of glucose. The present invention, however, is not limited thereto.

### Example 9

The present example examined that the drug of the present invention does not suppress lactate metabolism.

First, samples of 2-amino-1-cyclohexylethanol (2-amino-1-cyclohexyl EOH) and 2-amino-1-phenylethanol (2-amino-1-phenyl EOH) were prepared in the same manner as in Example 1. Four types of cells were used as in Example 8, and each of the cells was cultured under the same conditions as in Example 1. To these culture solutions, the samples were added so that the drug had a final concentration of 0.5 mg/ml. Thereafter, the culture time was lengthened to 2 to 3 days (48 to 72 hours) compared to Example 8. As a control 1, the culture was performed in the same manner except that distilled water to which the drug was not added was added to the culture solution instead of the sample. As a control 2, the culture was performed in the same manner as the control 2 except that a sample of biguanide prepared in the same manner as in Example 1 was added to the culture solution instead of the sample. Then, the lactate concentration in each sample was measured in the same manner as in Example 6. Each of the four cells was used for a total of four experiments.

The results are shown in FIG. 11. FIG. 11 is a graph showing the lactate concentration of each cell to which the drug was added. The value shown in the graph represents the mean value of the results of the four experiments. In FIG. 11, the vertical axis represents the lactate concentration (mmol/L) in the culture solution, and the horizontal axis represents the drug. As shown in FIG. 11, when 2 to 3 days culture was performed after addition of 0.5 mg/ml 2-amino-1-cyclohexylethanol and 2-amino-1-phenylethanol, the lactate concentrations were equivalent to that of control 1 (Control). On the other hand, in Control 2 (biguanide), when 2 to 3 days culture was performed after addition of biguanide, the lactate concentration was about 3.5 times (P<0.05) compared to Control 1. It is known that the lactate in the culture solution produced as a metabolite of the glycolysis is subsequently metabolized. Thus, since the lactate was metabolized to the same extent as control 1, it was examined that 2-amino-1-cyclohexylethanol and 2-amino-1-phenylethanol did not suppress the metabolism of lactate. In contrast, it was examined that biguanide suppressed the metabolism of lactate.

While the present invention has been described above with reference to illustrative embodiments and examples, the present invention is by no means limited thereto. Various changes and variations that may become apparent to those skilled in the art may be made in the configuration and specifics of the present invention without departing from the scope of the present invention.

This application claims priority from Japanese Patent Application No. 2018-143056 filed on Jul. 31, 2018, Japanese Patent Application No. 2018-181302 filed on Sep. 27, 2018, and PCT/JP2019/003915. The entire subject matter of the Japanese Patent Applications and the International Patent Application is incorporated herein by reference.

### INDUSTRIAL APPLICABILITY

As described above, according to the present invention, glucose consumption can be accelerated by including at least one selected from the group consisting of the compound represented by the chemical formula (1), tautomers and stereoisomers thereof, and salts thereof. Since the drug can accelerate the glucose consumption in this manner, for example, the drug can be used as a therapeutic agent for diabetes. Therefore, the present invention is extremely useful in the field of medicine and the like.

1-4. (canceled)

5. A glucose consumption accelerating method, comprising the step of:
administering a glucose consumption accelerator comprising at least one selected from the group consisting of 2-amino-1-cyclohexylethanol, 1-amino-2-propanol, 1,3-bis[tris(hydroxymethyl)methylamino]propane, N-cyclohexylethanolamine, diethanolamine, diethylamine, dipropylamine, morpholine, propylamine, triethanolamine, triethylamine, and trishydroxymethylaminomethane.

6-9. (canceled)

10. A glycolysis accelerating method comprising the step of:
administering a glycolysis accelerator comprising at least one selected from the group consisting of 2-amino-1-cyclohexylethanol, 1-amino-2-propanol, 1,3-bis[tris(hydroxymethyl)methylamino]propane, N-cyclohexylethanolamine, diethanolamine, diethylamine, dipropylamine, morpholine, propylamine, triethanolamine, triethylamine, and trishydroxymethylaminomethane.

11. The glucose consumption accelerating method according to claim 5, comprising the step of:
administering the glucose consumption accelerator comprising at least one selected from the group consisting of 2-amino-1-cyclohexylethanol, 1-amino-2-propanol, 1,3-bis[tris(hydroxymethyl)methylamino]propane, N-cyclohexylethanolamine, diethanolamine, diethylamine, dipropylamine, morpholine, propylamine, triethanolamine, triethylamine, and trishydroxymethylaminomethane as a main component.

12. The glucose consumption accelerating method according to claim 5, wherein
the glucose consumption accelerator further comprising:
an oral additive.

13. A treatment method of diabetes comprising the administering step in the glucose consumption accelerating method according to claim 5.

14. The glycolysis accelerating method according to claim 10, comprising the step of:
administering the glycolysis accelerator comprising at least one selected from the group consisting of 2-amino-1-cyclohexylethanol, 1-amino-2-propanol, 1,3-bis[tris(hydroxymethyl)methylamino]propane, N-cyclohexylethanolamine, diethanolamine, diethylamine, dipropylamine, morpholine, propylamine, triethanolamine, triethylamine, and trishydroxymethylaminomethane as a main component.

15. The glycolysis accelerating method according to claim 10, wherein
the glycolysis accelerator further comprising:
an oral additive.

16. A treatment method of diabetes comprising the administering step in the glycolysis accelerating method according to claim 10.
